(19) United States
(12) Patent Application Publication
(10) Pub. No.: US 2025/0228438 A1
(43) Pub. Date: Jul. 17, 2025

(54) TENSION ADJUSTMENT MECHANISM AND ENDOSCOPE DEVICE THEREOF

(71) Applicant: ALTEK BIOTECHNOLOGY CORPORATION, Hsinchu City (TW)

(72) Inventors: Wei-Ren Huang, Hsinchu City (TW); Pin-Chang Lai, Hsinchu City (TW); Hsi-Hsin Loo, Hsinchu City (TW)

(21) Appl. No.: 18/946,978

(22) Filed: Nov. 14, 2024

Related U.S. Application Data
(60) Provisional application No. 63/620,175, filed on Jan. 12, 2024.

(51) Int. Cl. A61B 1/005; A61B 1/04

(52) U.S. Cl. CPC A61B 1/0052; A61B 1/0057; A61B 1/04

(57) ABSTRACT

A tension adjustment mechanism includes a control handle, a control wheel, first and second adjustment screws rotatably screwed on the control wheel, and first and second control wires. The control handle has a handle body connected to a flexible tube of an endoscope device and a control lever operably inserted outside the handle body. The control wheel is rotatably disposed in the handle body and connected to the control lever. The first and second control wires movably pass through the flexible tube and are connected to the first and second adjustment screws respectively. Tensions of the first and second control wires are adjusted by rotating the first and second adjustment screws. When the control lever rotates the control wheel, the first and second control wires control a bending direction of the flexible tube together with rotation of the control wheel.

### CROSS REFERENCE TO RELATED APPLICATIONS

This application claims the benefit of U.S. Provisional Application No. 63/620,175, filed on Jan. 12, 2024. The content of the application is incorporated herein by reference.

### BACKGROUND OF THE INVENTION

### 1. Field of the Invention

The present invention relates to a tension adjustment mechanism and an endoscope device thereof, and more specifically, to a tension adjustment mechanism rotating adjustment screws to wrap control wires around the adjustment screws for adjusting tensions of the control wires on a control wheel and an endoscope device thereof.

### 2. Description of the Prior Art

An endoscope device is a long and flexible tube apparatus, and mainly includes an image capture device, a light source and a controllable bending tube. After the endoscope device is electrically connected to a monitor, organs in human's body can be imaging by the endoscope device and shown on the monitor, so that the endoscope device can be applied to medical inspection and treatment. In general, the endoscope device utilizes a control wheel to connect to two control wires threaded through the controllable bending tube, such that rotation of the control wheel can tighten one control wire and release the other control wire, thereby controlling a bending direction of the endoscope device (e.g., bending the endoscope device in an upward or downward direction) for the subsequent medical inspection.

However, during the assembly process of the control wheel and the control wire, the connection backlash between the control wire and the control wheel cannot be effectively eliminated, so as to cause the inability to accurately adjust the tension of the control wire, thus resulting in insufficient rotational travel of an insertion end of the controllable bending tube and affecting the deflection reliability of the endoscope device.

### SUMMARY OF THE INVENTION

The present invention provides a tension adjustment mechanism applied to wire tension adjustment of an endoscope device. The endoscope device includes a flexible tube and an endoscope lens module. A distal end of the flexible tube is connected to the endoscope lens module. The tension adjustment mechanism includes a control handle, a control wheel, a first adjustment screw, a second adjustment screw, a first control wire, and a second control wire. The control handle has a handle body and a control lever. The handle body is connected to a proximal end of the flexible tube. The control lever is operably inserted outside the handle body. The control wheel is rotatably disposed in the handle body and connected to the control lever to rotate relative to the handle body by operating the control lever. The control wheel has a circumferential guide groove, a first guide hole, and a second guide hole, and the first guide hole and the second guide hole are respectively located at two ends of the circumferential guide groove. The first adjustment screw is rotatably screwed on the control wheel. The second adjustment screw is rotatably screwed on the control wheel. The first control wire movably passes through the flexible tube and passes through the first guide hole along the circumferential guide groove to connect to the first adjustment screw, wherein a tension of the first control wire is adjusted by rotating the first adjustment screw to wrap the first control wire around the first adjustment screw. The second control wire movably passes through the flexible tube and passes through the second guide hole along the circumferential guide groove to connect to the second adjustment screw, wherein a tension of the second control wire is adjusted by rotating the second adjustment screw to wrap the second control wire around the second adjustment screw. When the control lever rotates the control wheel, the first control wire and the second control wire control a bending direction of the flexible tube together with rotation of the control wheel.

The present invention further provides an endoscope device including a flexible tube, an endoscope device, and a tension adjustment mechanism. The endoscope lens module is connected to a distal end of the flexible tube. The tension adjustment mechanism includes a control handle, a control wheel, a first adjustment screw, a second adjustment screw, a first control wire, and a second control wire. The control handle has a handle body and a control lever. The handle body is connected to a proximal end of the flexible tube. The control lever is operably inserted outside the handle body. The control wheel is rotatably disposed in the handle body and connected to the control lever to rotate relative to the handle body by operating the control lever. The control wheel has a circumferential guide groove, a first guide hole, and a second guide hole, and the first guide hole and the second guide hole are respectively located at two ends of the circumferential guide groove. The first adjustment screw is rotatably screwed on the control wheel. The second adjustment screw is rotatably screwed on the control wheel. The first control wire movably passes through the flexible tube and passes through the first guide hole along the circumferential guide groove to connect to the first adjustment screw, wherein a tension of the first control wire is adjusted by rotating the first adjustment screw to wrap the first control wire around the first adjustment screw. The second control wire movably passes through the flexible tube and passes through the second guide hole along the circumferential guide groove to connect to the second adjustment screw, wherein a tension of the second control wire is adjusted by rotating the second adjustment screw to wrap the second control wire around the second adjustment screw. When the control lever rotates the control wheel, the first control wire and the second control wire control a bending direction of the flexible tube together with rotation of the control wheel.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of an endoscope device according to an embodiment of the present invention.

FIG. 2 is an enlarged diagram of a tension adjustment mechanism in FIG. 1.

FIG. 3 is an enlarged diagram of a control wheel in FIG. 2.

FIG. 4 is an enlarged diagram of a first adjustment screw in FIG. 2.

### DETAILED DESCRIPTION

The present invention will now be described more specifically with reference to the following embodiments and the accompanying drawings. Other advantages and effects of the present invention can be easily understood by a person ordinarily skilled in the art in view of the detailed descriptions and the accompanying drawings. The present invention can be implemented or applied to other different embodiments. Certain aspects of the present invention are not limited by the particular details of the examples illustrated herein. Without departing from the spirit and scope of the present invention, the present invention will have other modifications and changes. It should be understood that the appended drawings are not necessarily drawn to the scale and configuration of each component (e.g., a handle design, sizes of a flexible tube, an endoscope lens module, and a tension adjustment mechanism, etc.) in the drawings is merely illustrative, not presenting an actual condition of the embodiments. In the following detailed description of the preferred embodiments, reference is made to the accompanying drawings which form a part hereof, and in which is shown by way of illustration specific embodiments in which the invention may be practiced. In this regard, directional terminology, such as “up”, “down”, “left”, “right”, “front”, “back”, etc., is used with reference to the orientation of the drawing(s) being described. Also, the term “connect” is intended to mean either an indirect or direct electrical/mechanical connection. Thus, if a first device is connected to a second device, that connection may be through a direct electrical/mechanical connection, or through an indirect electrical/mechanical connection via other devices and connections.

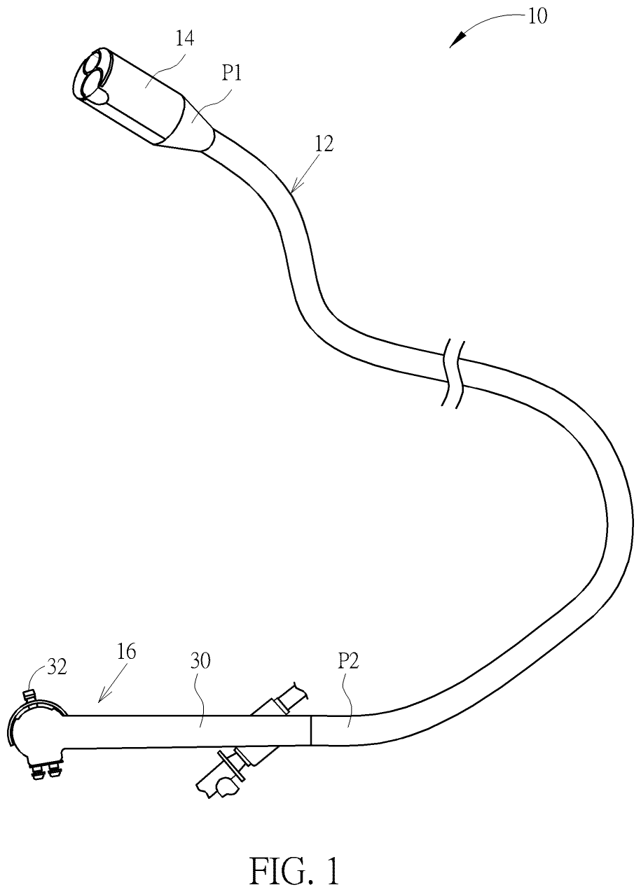

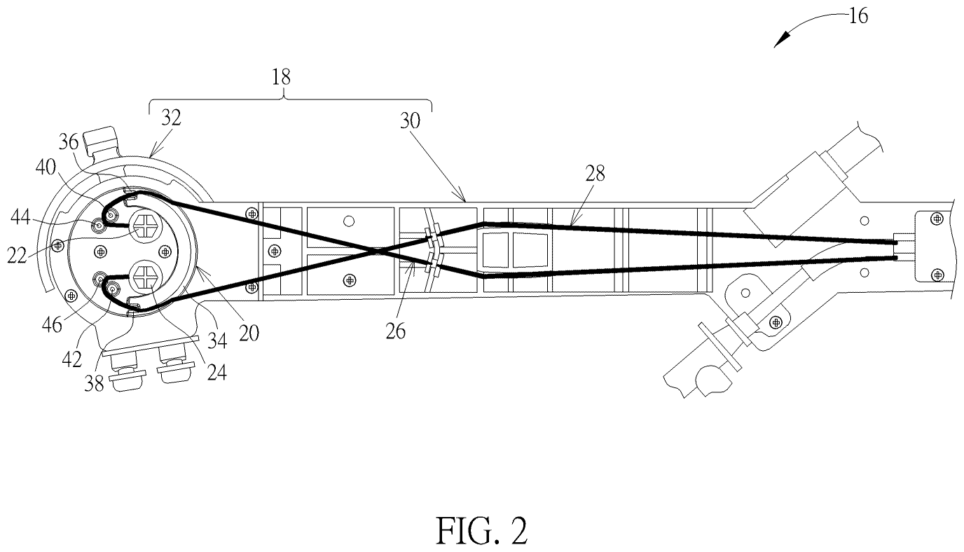

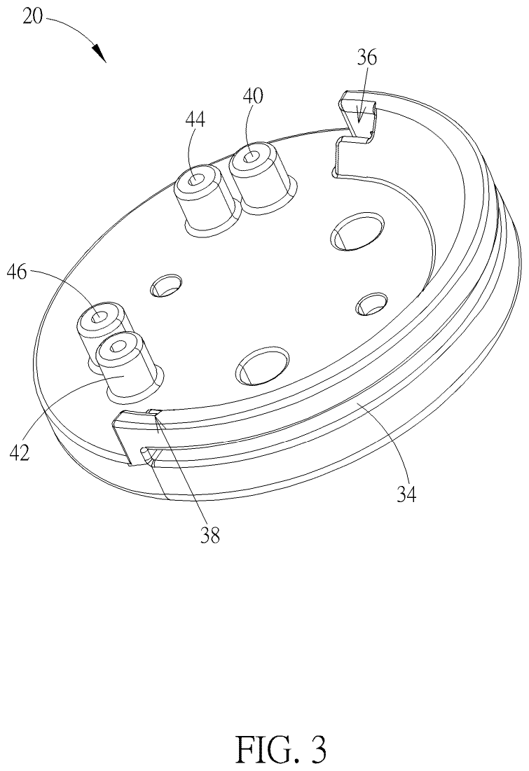

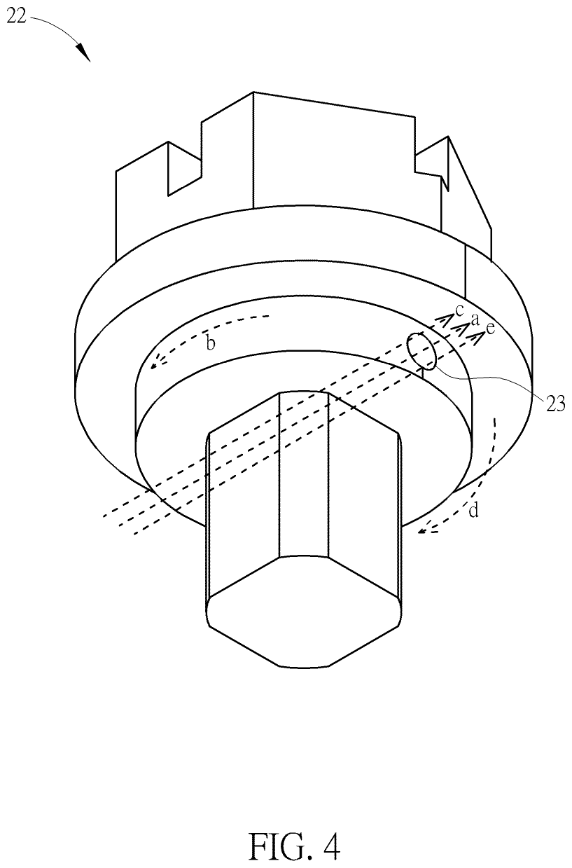

Please refer to FIGS. 1-4. FIG. 1 is a diagram of an endoscope device 10 according to an embodiment of the present invention. FIG. 2 is an enlarged diagram of a tension adjustment mechanism 16 in FIG. 1. FIG. 3 is an enlarged diagram of a control wheel 20 in FIG. 2. FIG. 4 is an enlarged diagram of a first adjustment screw 22 in FIG. 2. For clearly showing an internal configuration of the tension adjustment mechanism 16, the tension adjustment mechanism 16 is presented in a cross-sectional view in FIG. 2. As shown in FIGS. 1-4, the endoscope device 10 includes a flexible tube 12, an endoscope lens module 14, and the tension adjustment mechanism 16, and the tension adjustment mechanism 16 includes a control handle 18, the control wheel 20, the first adjustment screw 22, a second adjustment screw 24, a first control wire 26, and a second control wire 28. The flexible tube 12 could be preferably made of plastic material (e.g., PP (Polypropylene) or POM (Polyoxymethylene)) and formed by a plastic mold injection process. The endoscope lens module 14 is connected to a distal end P1 of the flexible tube 12, the tension adjustment mechanism 16 is disposed on a proximal end P2 of the flexible tube 12, and the control wheel 20 is connected to the endoscope lens module 14 via the first control wire 26 and the second control wire 28 passing through the flexible tube 12. In such a manner, rotation of the control wheel 20 can tighten one control wire and release the other control wire, thereby controlling a bending direction of the flexible tube 12 to operate the endoscope lens module 14 to perform endoscope imaging (e.g., imaging organs in human's body) for the subsequent medical inspection. As for the wire controlling design of the control wheel 20 and the imaging design of the endoscope lens module 14, the related description is commonly seen in the prior art and omitted herein.

More detailed description for a tension adjustment design of the tension adjustment mechanism 16 is provided as follows with reference to FIGS. 1-4. The control handle 18 has a handle body 30 and a control lever 32. The handle body 30 is connected to the proximal end P2 of the flexible tube 12, and the control lever 32 is operably inserted outside the handle body 30. The control wheel 20 is rotatably disposed in the handle body 30 and connected to the control lever 32 to rotate relative to the handle body 30 by operating the control lever 32. The related description for the operation design of the control lever 32 rotating the control wheel 20 is omitted herein for simplicity since it is commonly seen in the prior art. The control wheel 20 has a circumferential guide groove 34, a first guide hole 36, and a second guide hole 38, and the first guide hole 36 and the second guide hole 38 are respectively located at two ends of the circumferential guide groove 34. The first adjustment screw 22 and the second adjustment screw 24 are rotatably screwed on the control wheel 20. The first control wire 26 movably passes through the flexible tube 12 and passes through the first guide hole 36 along the circumferential guide groove 34 to connect to the first adjustment screw 22, and the second control wire 28 movably passes through the flexible tube 12 and passes through the second guide hole 38 along the circumferential guide groove 34 to connect to the second adjustment screw 24. The first control wire 26 and the second control wire are 28 are preferably arranged in a crossed configuration within the handle body 30, but not limited thereto, meaning that the present invention could adopt other wire arrangement configuration in another embodiment, such as a wire parallel configuration.

To be more specific, as shown in FIGS. 2 and 3, for guiding the first control wire 26 and the second control wire 28 to connect to the first adjustment screw 22 and the second adjustment screw 24 more smoothly and ensuring that the first control wire 26 and the second control wire 28 can maintain sufficient tensions, in this embodiment, the control wheel 20 could further have a first guide pillar 40, a second guide pillar 42, a third guide pillar 44, and a fourth guide pillar 46. The third guide pillar 44 is located adjacent to the first guide pillar 40 for guiding the first control wire 26 to pass between the first guide pillar 40 and the third guide pillar 44 to connect to the first adjustment screw 22. The fourth guide pillar 46 is located adjacent to the second guide pillar 42 for guiding the second control wire 28 to pass between the second guide pillar 42 and the fourth guide pillar 46 to connect to the second adjustment screw 24. Moreover, the present invention could adopt a screw hole design to further enhance the connection strength and stability of the first control wire 26 and the second control wire 28 on the control wheel 20. For example, as shown in FIG. 4, the first adjustment screw 22 could have a threading hole 23, and the first control wire 26 can pass through the threading hole 23 and be wrapped around the first adjustment screw 22 (e.g., along wrapping routes a, b, c, d, and e sequentially, but not limited thereto). As for the wire wrapping design of the second control wire 28 on the second adjustment screw 24, the related description could be reasoned by analogy according to FIG. 4 and omitted herein.

Via the aforesaid design, after the first control wire 26 movably passes through the flexible tube 12, passes through the first guide hole 36 along the circumferential guide groove 34, and then passes between the first guide pillar 40 and the third guide pillar 44 to connect to the first adjustment screw 22, a user can rotate the first adjustment screw 22 via a tool (e.g., a screw driver) to wrap the first control wire 26 around the first adjustment screw 22 tightly for properly adjusting a tension of the first control wire 26, so as to eliminate a connection backlash between the first control wire 26 and the control wheel 20. Similarly, after the second control wire 28 movably passes through the flexible tube 12, passes through the second guide hole 38 along the circumferential guide groove 34, and then passes between the second guide pillar 42 and the fourth guide pillar 46 to connect to the second adjustment screw 24, the user can also rotate the second adjustment screw 24 to wrap the second control wire 28 around the second adjustment screw 24 tightly for properly adjusting a tension of the second control wire 28, so as to eliminate a connection backlash between the second control wire 28 and the control wheel 20. In such a manner, when the user operates the control lever 32 to rotate the control wheel 20, the first control wire 26 and the second control wire 28 with appropriate tensions can control a bending direction of the flexible tube 12 without insufficient rotational travel of the distal end P1 of the flexible tube 12, so that the user can operate the endoscope lens module 14 to perform precise endoscope imaging (e.g., imaging organs in human's body) for the subsequent medical inspection.

To be noted, the present invention could apply a glue fixing design to connection between the control wire and the control wheel. For example, after the aforesaid tension adjustment process is completed, the first control wire 26 could be glued to at least one of the first guide hole 36, the first guide pillar 40, the third guide pillar 44, and the first adjustment screw 22, so as to improve the connection strength and stability of the first control wire 26 on the control wheel 20. Similarly, after the aforesaid tension adjustment process is completed, the second control wire 28 could be glued to at least one of the second guide hole 38, the second guide pillar 42, the fourth guide pillar 46, and the second adjustment screw 24, so as to improve the connection strength and stability of the second control wire 28 on the control wheel 20.

In addition, it should be mentioned that the aforesaid guide pillars could be selectively omitted for simplifying the wire connection design of the present invention. For example, in another embodiment without the third guide pillar 44 and the fourth guide pillar 46, the first control wire 26 could be only routed around the first guide pillar 40 to connect to the first adjustment screw 22, and the second control wire 28 could be only routed around the second guide pillar 42 to connect to the second adjustment screw 24. In yet another embodiment without the first guide pillar 40, the second guide pillar 42, the third guide pillar 44 and the fourth guide pillar 46, the first control wire 26 could be directly connected to the first adjustment screw 22 after passing through the first guide hole 36 along the circumferential guide groove 34, and the second control wire 28 could be directly connected to the second adjustment screw 24 after passing through the second guide hole 38 along the circumferential guide groove 34.

Compared with the prior art, the present invention adopts the screw adjustment design in which the tension of the control wire can be properly adjusted by rotating the adjustment screw to wrap the control wire around the adjustment screw, for ensuring that the connection backlash between the control wire and the control wheel can be eliminated. In such a manner, the present invention can efficiently solve the prior art problem that idle travel of the control wheel rotation occurs due to the connection backlash between the control wire and the control wheel and the inability to accurately adjust the tension of the control wire, so as to greatly improve the deflection reliability and operational accuracy of the endoscope device.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A tension adjustment mechanism applied to wire tension adjustment of an endoscope device, the endoscope device comprising a flexible tube and an endoscope lens module, a distal end of the flexible tube being connected to the endoscope lens module, the tension adjustment mechanism comprising:
a control handle having a handle body and a control lever, the handle body being connected to a proximal end of the flexible tube, the control lever being operably inserted outside the handle body;
a control wheel rotatably disposed in the handle body and connected to the control lever to rotate relative to the handle body by operating the control lever, the control wheel having a circumferential guide groove, a first guide hole, and a second guide hole, and the first guide hole and the second guide hole being respectively located at two ends of the circumferential guide groove;
a first adjustment screw rotatably screwed on the control wheel;
a second adjustment screw rotatably screwed on the control wheel;
a first control wire movably passing through the flexible tube and passing through the first guide hole along the circumferential guide groove to connect to the first adjustment screw, wherein a tension of the first control wire is adjusted by rotating the first adjustment screw to wrap the first control wire around the first adjustment screw; and
a second control wire movably passing through the flexible tube and passing through the second guide hole along the circumferential guide groove to connect to the second adjustment screw, wherein a tension of the second control wire is adjusted by rotating the second adjustment screw to wrap the second control wire around the second adjustment screw;
wherein when the control lever rotates the control wheel, the first control wire and the second control wire control a bending direction of the flexible tube together with rotation of the control wheel.

2. The tension adjustment mechanism of claim 1, wherein the first control wire and the second control wire are arranged in a crossed or parallel configuration within the handle body.

3. The tension adjustment mechanism of claim 1, wherein the first control wire and the second control wire are respectively glued to the first guide hole and the second guide hole after the tension of the first control wire and the tension of the second control wire are respectively adjusted by the first adjustment screw and the second adjustment screw.

4. The tension adjustment mechanism of claim 1, wherein the control wheel further has a first guide pillar and a second guide pillar, the first control wire passes through the first guide hole and is routed around the first guide pillar to connect to the first adjustment screw, and the second control wire passes through the second guide hole and is routed around the second guide pillar to connect to the second adjustment screw.

5. The tension adjustment mechanism of claim 4, wherein the first control wire and the second control wire are respectively glued to the first guide pillar and the second guide pillar after the tension of the first control wire and the tension of the second control wire are respectively adjusted by the first adjustment screw and the second adjustment screw.

6. The tension adjustment mechanism of claim 4, wherein the control wheel further has a third guide pillar and a fourth guide pillar, the third guide pillar is located adjacent to the first guide pillar for guiding the first control wire to pass between the first guide pillar and the third guide pillar to connect to the first adjustment screw, and the fourth guide pillar is located adjacent to the second guide pillar for guiding the second control wire to pass between the second guide pillar and the fourth guide pillar to connect to the second adjustment screw.

7. The tension adjustment mechanism of claim 6, wherein the first control wire is glued to the first guide pillar and the third guide pillar after the tension of the first control wire is adjusted by the first adjustment screw, and the second control wire is glued to the second guide pillar and the fourth guide pillar after the tension of the second control wire is adjusted by the second adjustment screw.

8. The tension adjustment mechanism of claim 1, wherein the first control wire and the second control wire are respectively glued to the first adjustment screw and the second adjustment screw after the tension of the first control wire and the tension of the second control wire are respectively adjusted by the first adjustment screw and the second adjustment screw.

9. The tension adjustment mechanism of claim 1, wherein the first adjustment screw has a threading hole, and the first control wire passes through the threading hole and is wrapped around the first adjustment screw.

10. An endoscope device comprising:
a flexible tube;
an endoscope lens module connected to a distal end of the flexible tube; and
a tension adjustment mechanism comprising:
a control handle having a handle body and a control lever, the handle body being connected to a proximal end of the flexible tube, the control lever being operably inserted outside the handle body;
a control wheel rotatably disposed in the handle body and connected to the control lever to rotate relative to the handle body by operating the control lever, the control wheel having a circumferential guide groove, a first guide hole, and a second guide hole, and the first guide hole and the second guide hole being respectively located at two ends of the circumferential guide groove;
a first adjustment screw rotatably screwed on the control wheel;
a second adjustment screw rotatably screwed on the control wheel;
a first control wire movably passing through the flexible tube and passing through the first guide hole along the circumferential guide groove to connect to the first adjustment screw, wherein a tension of the first control wire is adjusted by rotating the first adjustment screw to wrap the first control wire around the first adjustment screw; and
a second control wire movably passing through the flexible tube and passing through the second guide hole along the circumferential guide groove to connect to the second adjustment screw, wherein a tension of the second control wire is adjusted by rotating the second adjustment screw to wrap the second control wire around the second adjustment screw;
wherein when the control lever rotates the control wheel, the first control wire and the second control wire control a bending direction of the flexible tube together with rotation of the control wheel.

11. The endoscope device of claim 10, wherein the first control wire and the second control wire are arranged in a crossed or parallel configuration within the handle body.

12. The endoscope device of claim 10, wherein the first control wire and the second control wire are respectively glued to the first guide hole and the second guide hole after the tension of the first control wire and the tension of the second control wire are respectively adjusted by the first adjustment screw and the second adjustment screw.

13. The endoscope device of claim 10, wherein the control wheel further has a first guide pillar and a second guide pillar, the first control wire passes through the first guide hole and is routed around the first guide pillar to connect to the first adjustment screw, and the second control wire passes through the second guide hole and is routed around the second guide pillar to connect to the second adjustment screw.

14. The endoscope device of claim 13, wherein the first control wire and the second control wire are respectively glued to the first guide pillar and the second guide pillar after the tension of the first control wire and the tension of the second control wire are respectively adjusted by the first adjustment screw and the second adjustment screw.

15. The endoscope device of claim 13, wherein the control wheel further has a third guide pillar and a fourth guide pillar, the third guide pillar is located adjacent to the first guide pillar for guiding the first control wire to pass between the first guide pillar and the third guide pillar to connect to the first adjustment screw, and the fourth guide pillar is located adjacent to the second guide pillar for guiding the second control wire to pass between the second guide pillar and the fourth guide pillar to connect to the second adjustment screw.

16. The endoscope device of claim 15, wherein the first control wire is glued to the first guide pillar and the third guide pillar after the tension of the first control wire is adjusted by the first adjustment screw, and the second control wire is glued to the second guide pillar and the fourth guide pillar after the tension of the second control wire is adjusted by the second adjustment screw.

17. The endoscope device of claim 10, wherein the first control wire and the second control wire are respectively glued to the first adjustment screw and the second adjustment screw after the tension of the first control wire and the tension of the second control wire are respectively adjusted by the first adjustment screw and the second adjustment screw.

18. The endoscope device of claim 10, wherein the first adjustment screw has a threading hole, and the first control wire passes through the threading hole and is wrapped around the first adjustment screw.
